(19) United States
(12) Patent Application Publication
(10) Pub. No.: US 2016/0074023 A1
(43) Pub. Date: Mar. 17, 2016

(54) MEDICAL MATERIAL

(71) Applicant: GUNZE LIMITED, Ayabe-shi, Kyoto (JP)

(72) Inventors: Yuki SAKAMOTO, Ayabe-shi (JP); Kensuke TAKI, Ayabe-shi (JP); Hidetaka NAKAYAMA, Ayabe-shi (JP); Noriko KAWADA, Ayabe-shi (JP)

(73) Assignee: GUNZE LIMITED, Ayabe-shi, Kyoto (JP)

(21) Appl. No.: 14/784,465

(22) PCT Filed: Apr. 22, 2014
(86) PCT No.: PCT/JP2014/061317

§ 371 Date: Oct. 14, 2015

(30) Foreign Application Priority Data
May 15, 2013 (JP) 2013-103268
Feb. 19, 2014 (JP) 2014-029675

(51) Int. Cl. A61B 17/00; A61B 17/12

(52) U.S. Cl. CPC A61B 17/0057; A61B 17/12113; A61B 17/12177; A61B 2017/00004; A61B 2017/00575; A61B 2017/00592; A61B 2017/00606; A61B 2017/00623; A61B 2017/00942

(57) ABSTRACT

There is provided a defect hole closing material which achieves minimally invasive medical treatment for Atrial Septal Defect, and poses little possibility of occurrence of a failure in the body in some distant future.

The defect hole closing material (100) comprises two bioabsorbable material-made cylindrical bodies (the first cylindrical section (110) and the second cylindrical section (120)) having a mesh-like structure in the form of an hourglass shape, a numeral “8” shape, a double spindle shape, or a peanut shape. With the first end (112) kept held securely, the second end, or the other end (122) is pulled toward the first end (112) by operating the string (140), whereupon the cylinder diameter is increased, and also the first cylindrical section (110) and the second cylindrical section (120) move close to each other with respect to the substantially central section (130) serving as a center.

### TECHNICAL FIELD

The present invention relates to a medical material for treatment of a defect hole, an aneurysm, and so forth developed in biological tissue, and more particularly to a medical material which is set in a catheter, is delivered to a body area to be treated through blood vessel, and is left to indwell in a living body.

### BACKGROUND ART

The heart of a human being is divided into right and left chambers by an organ called septum, and each of the right and left chambers has cardiac atrium and cardiac ventricle, and more specifically, the heart is composed of two atria and two ventricles, namely right atrium, right ventricle, left atrium, and left ventricle. As a disease of the heart having such a structure, there is known ASD (Atrial Septal Defect), which is a cardiac disorder caused by a developmental difficulty in a fetal stage, characterized by a congenital absence called a defect hole developed in atrial septum acting as a partition between the right atrium and the left atrium.

As attempts to treat ASD, the following two medical procedures are practiced, i.e. an open-heart surgery, and catheterization which is performed by means of a closure plug without thoracotomy.

In the case of surgery (patching operation), thoracotomy is performed using an artificial heart-lung machine to close a defect hole by a patch. In the case of catheterization, a closure plug is set in a catheter, and the catheter is inserted into blood vessel and is delivered to a target position (defect hole), and subsequently the closure plug is released so as to indwell in the body. In catheterization procedure, hole closure is effected by inserting a small jig (device) folded in strip form called a closure plug into the body from a vein at the base of human leg (femoral vein), and delivering the closure plug to the position of a hole developed in the atrial septum; that is, thoracotomy is not performed. The positive side of the catheterization procedure is that treatment can be achieved simply by making a minute skin incision (which measures only a few millimeters) at the base of human leg (groin) which is an inconspicuous body area without performing thoracotomy that necessitates induction of general anesthesia.

In Japanese Unexamined Patent Publication JP-A 2008-512139 (Patent literature 1), there is disclosed an assembly for use in ASD catheterization procedure (closure plug). This assembly seals a passageway (defect hole) in the heart hermetically. The assembly comprises: a closure device for hermetically sealing a heart passageway including a first anchor used for placement in the vicinity of a first end of the passageway, a second anchor used for placement in the vicinity of a second end of the passageway, and a flexible extension material, which runs through the passageway, used for connection with the first and second anchors, the second anchor being movable relative to the flexible extension material so that the length of the flexible extension material between the first and second anchors can be changed; and a supply system for delivering the closure device to the heart passageway, in which a supply device is designed to move within the inner cavity of a guide catheter, and a wire for controlling the movement of the second anchor along the flexible extension material is included.

According to the disclosure of Patent literature 1, a PFO (Patent Oval Foramen) closure device (closure plug) comprises: a left atrium anchor; a right atrium anchor; a tether; and a lock, and, the left atrium anchor, the right atrium anchor which is coupled to the left atrium anchor via the tether, and the lock remain in the heart to seal PFO hermetically.

### PRIOR ART REFERENCE

### Patent Literature

Patent literature 1: Japanese Unexamined Patent Publication JP-A 2008-512139

### SUMMARY OF THE INVENTION

### Problems to be Solved by the Invention

The patching operation entails the use of an artificial heart-lung machine and poses high invasiveness, with consequent prolonged hospitalization. In contrast, the catheterization procedure does not necessitate an artificial heart-lung machine and is minimally invasive, which is conducive to reduction in the length of hospitalization as is desirable.

As disclosed in Patent literature 1, the left atrium anchor and the right atrium anchor remain in the heart. The left atrium anchor and the right atrium anchor include one or more arms, and the arm extends radially outwardly from a hub. The arm is preferably formed of a rolled sheet made of a nickel-titanium two-component alloy. A defect hole is closed by expanding the left atrium anchor and the right atrium anchor within a living body, but, in this case, once the expansion of the anchor is started, it is difficult to allow the anchor to recover its original configuration. Therefore, as disclosed in Patent literature 1, the anchor has to be folded by means of a special-purpose retrievable device which has a complicated structure and is difficult to operate from outside the living body.

However, in the event of an accident, for example, in the event that the anchor is accidentally caught in biological tissue within the atrium with consequent occurrence an injury, there may be a case where there is not enough time to fold the anchor properly by such a retrievable device. In this case, there is no other choice but to start thoracotomy immediately. After all, a patient has to undergo thoracotomy which is a highly invasive surgery.

Another problem is that there arises a concern about occurrence of a failure in the body in some distant future because of the lifelong existence of a metal-made defect hole closure plug within the body.

In addition to such a defect hole, as a disease to which catheterization procedure is applicable, there is known an aneurysm which is developed in some part of the living body. The aforestated problems hold true for aneurysm treatment.

The present invention has been developed in view of the problems associated with conventional art as mentioned supra, and accordingly its object is to provide a medical material which is readily releasable and left to indwell in a target area to be treated within a living body, helps facilitate minimally invasive catheterization procedure without an intricate mechanism, and poses little possibility of occurrence of a failure in the body in some distant future even if it remains in the body.

### Means for Solving the Problem

In order to accomplish the above object, the following technical means is adopted for the implementation of a medical material pursuant to the present invention.

That is, a medical material pursuant to the present invention comprises a cylindrical body of mesh-like structure made of a filamentary material, and has a configuration such that a cylinder diameter of a substantially central section of said cylindrical body is made smaller than a cylinder diameter of other section of said cylindrical body by constricting said substantially central section.

In the invention, it is preferable that the cylinder diameter of said other section can be increased by, while securely holding a first end, which is one end of said medical material in a direction of length of the cylindrical body, pulling a second end, which is the other end, toward the first end.

Moreover, it is preferable that, with respect to said substantially central section serving as a center, there are provided a first cylindrical section located toward the first end and a second cylindrical section located toward the second end, and that said first cylindrical section and said second cylindrical section are caused to move close to each other with respect to said substantially central section serving as a center by pulling the second end, or equivalently the other end, of said cylindrical body toward the first end of said cylindrical body while holding the first end securely.

Moreover, it is preferable that there is further provided a string which is passed into said cylindrical body so as to be directed to and engage with said second end, and then run up to said first end.

Moreover, it is preferable that, with respect to said substantially central section serving as a center, there are provided a first cylindrical section located toward the first end and a second cylindrical section located toward the second end, and that a cylinder diameter of said first cylindrical section can be increased by, while securely holding the first end of said cylindrical body, pulling a substantially central section-side end of said first cylindrical section toward the first end.

Moreover, it is preferable that there is further provided a string which is passed into said cylindrical body so as to be directed to and engage with said substantially central section-side end of said first

Moreover, it is preferable that, with respect to said substantially central section serving as a center, there are provided a first cylindrical section located toward the first end and a second cylindrical section located toward the second end, and that there is provided a string which is passed into said cylindrical body from said first end, is directed to said second end, engages with said second end, runs through the interior of said cylindrical body toward said first end, engages with said first end, runs through the interior of said cylindrical body once again toward said second end, engages with said second end once again, and runs through the interior of the cylindrical body once again toward said first end.

Moreover, it is preferable that, with respect to said substantially central section serving as a center, there are provided a first cylindrical section located toward the first end and a second cylindrical section located toward the second end, and that there is provided a string which is passed into said cylindrical body from said first end, is directed to said second end, engages with said second end, runs through the interior of said cylindrical body toward said substantially central section, engages with said substantially central section, runs through the interior of said cylindrical body once again toward said second end, engages with said second end once again, and runs through the interior of the cylindrical body once again toward said first end.

Moreover, it is preferable that said configuration can be defined by an hourglass shape, a numeral “8” shape, or a double spindle shape.

Moreover, it is preferable that said filamentary material is a bioabsorbable material.

Moreover, it is preferable that a porous cylindrical layer, which is composed of any one of a nonwoven cloth, a sponge, a film, and a composite of them that are made of a bioabsorbable material, is placed on the inner surface of said cylindrical body.

### Advantageous Effects of the Invention

According to the present invention, the medical material is readily releasable and left to indwell in a target area to be treated within a living body, and allows minimally invasive catheterization procedure with simple operation without an intricate mechanism. In addition, the medical material of the present invention poses little possibility of occurrence of a failure in the body in some distant future even if it remains in the body.

### BRIEF DESCRIPTION OF DRAWINGS

FIG. 1 is a general perspective view of a defect hole closing material in accordance with one embodiment of the present invention.

FIG. 2A is a fragmentary side view of the defect hole closing material shown in FIG. 1.

FIG. 2B is a sectional view taken along the line A-A shown in FIG. 1.

FIG. 3 is a conceptual diagram illustrating the case of using the defect hole closing material shown in FIG. 1 in ASD catheterization procedure.

FIG. 4 is an enlarged view of Part B depicted in FIG. 3, illustrating a procedural step of catheterization procedure (1).

FIG. 5 is an enlarged view of Part B depicted in FIG. 3, illustrating a procedural step of catheterization procedure (2).

FIG. 6 is an enlarged view of Part B depicted in FIG. 3, illustrating a procedural step of catheterization procedure (3).

FIG. 7 is an enlarged view of Part B depicted in FIG. 3, illustrating a procedural step of catheterization procedure (4).

FIG. 8 is an enlarged view of Part B depicted in FIG. 3, illustrating a procedural step of catheterization procedure (5).

FIG. 9 is a view illustrating a procedural step of catheterization for aneurysm treatment (1).

FIG. 10 is a view illustrating a procedural step of catheterization for aneurysm treatment (2).

FIG. 11 is a view illustrating a procedural step of catheterization for aneurysm treatment (3).

FIG. 12 is a general perspective view of an example of modified form of the defect hole closing material pursuant to the present invention.

### MODES FOR CARRYING OUT THE INVENTION

Hereinafter, a medical material pursuant to the present invention will be described in detail with reference to drawings. While the following description deals with a defect hole closing material for use in catheterization procedure by way of example of the medical material of the present invention, the present invention is suitably applicable also to the closure of other opening hole or passageway, for example, an opening hole in the heart such as Ventricular septal defect or Patent ductus arteriosus, and an opening hole or passageway in other part of a living body (for example, stomach) such as Arteriovenous fistula. Accordingly, the use of a defect hole closing material in accordance with an embodiment of the present invention is not limited to the closure of a hole arising from ASD. Particularly, as will hereafter be described by way of another usage mode, the defect hole closing material is applicable to catheterization procedure for aneurysm treatment, and, in addition to that, it is also applicable to catheterization procedure to treat other diseases.

Moreover, although, in the following embodiment, the mesh-like structure of a defect hole closing material 100 will be illustrated as being obtained by knitting bioabsorbable fiber (an example of filamentary materials), the present invention is not so limited. It is essential only that the defect hole closing material be capable of catheterization to close a defect hole developed in a living body, and therefore, its mesh-like structure may be made of a filamentary material other than bioabsorbable fiber so long as the material has a first characteristic and a second characteristic and exhibits a first effect and a second effect as will hereafter be described. Such a filamentary material should preferably have a certain degree of hardness for the sake of shape retainability.

### [Constitution]

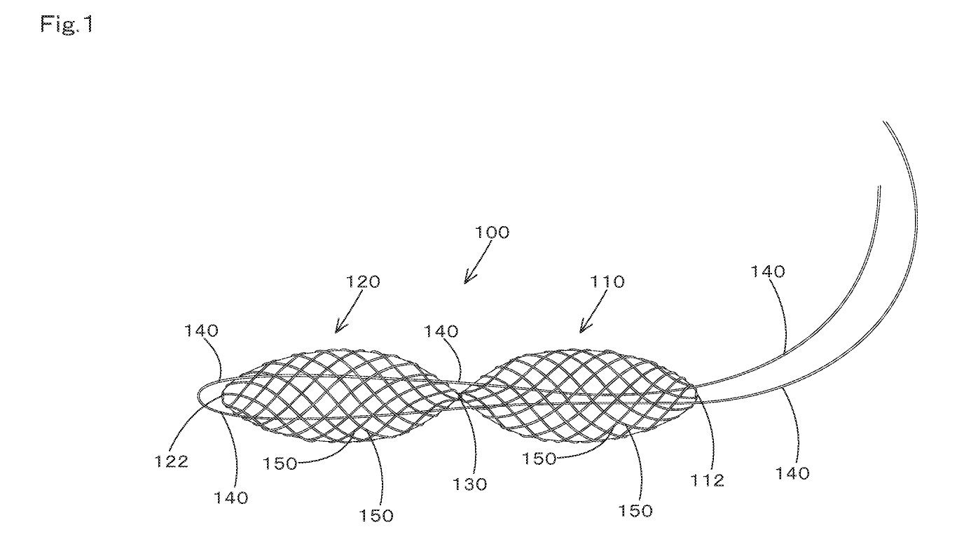

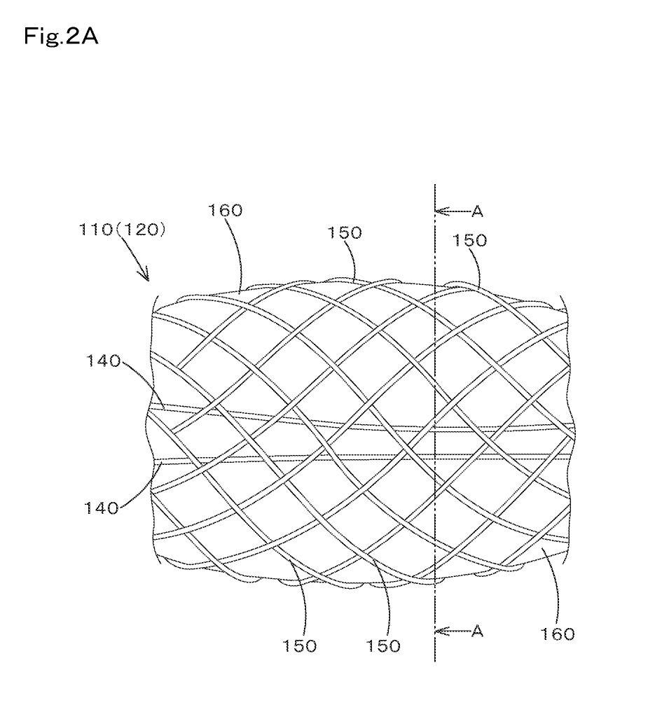

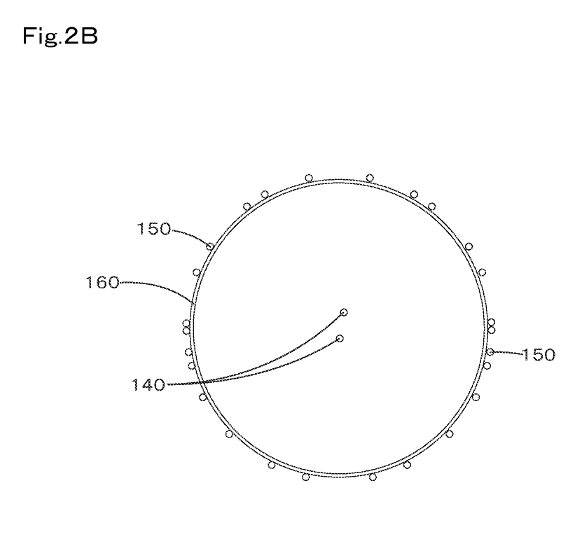

FIG. 1 shows a general perspective view of the defect hole closing material 100 (closure plug) of the present embodiment; FIG. 2A shows a fragmentary side view of the defect hole closing material 100; and FIG. 2B is a sectional view taken along the line A-A shown in FIG. 2A. Note that, in FIG. 2B which is a sectional view of the defect hole closing material 100, only the sections of a string 140, bioabsorbable fiber 150, and a porous cylindrical layer 160 are illustrated, and, a meshed pattern of the bioabsorbable fiber 150 that is viewable from a direction indicated by an arrow A is omitted. Moreover, in FIGS. 1, 2A, and 2B, in order to simplify an understanding of the presence of the string 140 and the meshed pattern of the bioabsorbable fiber 150, the porous cylindrical layer 160 is illustrated as a transparent material.

As shown in these drawings, the defect hole closing material 100 comprises two bioabsorbable material-made cylindrical bodies (a first cylindrical section 110 and a second cylindrical section 120) having a mesh-like structure in the form of, for example, an hourglass shape, a shape like a numeral “8”, a double spindle shape (a shape defined by two continuously arranged slim rodlike spindle objects, each of which is thicker at the midportion than at each end), or a peanut shape (a shape defined by the contour of a peanut shell containing two peanuts). The defect hole closing material 100 designed in such a form has its substantially central section 130 constricted so that the cylinder diameter of the substantially central section 130 is smaller than the cylinder diameter of other section of the defect hole closing material 100. That is, the defect hole closing material 100 comprises: the substantially central section 130 located centrally thereof; the first cylindrical section 110 located at a first end 112 side thereof; and the second cylindrical section 120 located at a second end 122 side thereof.

Although there is no particular limitation, it is advisable that the defect hole closing material 100 comprising two cylindrical bodies in the form of an hourglass shape, a numeral “8” shape, a double spindle shape, or a peanut shape is produced by forming a cylindrical body which has a substantially uniform diameter by knitting the first cylindrical section 110 and the second cylindrical section 120 in a single-piece form, and subsequently constricting the cylindrical body at its substantially central section 130 by a string made of the same material as that used for the first cylindrical section 110 and the second cylindrical section 120.

Moreover, the defect hole closing material 100 includes a string 140 which is passed into the cylindrical body so as to be directed to and engage with the second end 122 (to be hitched on a loop at the second end 122) and then run up to the first end 112. With use of the string 140, while the first end 112, which is lengthwise one end of the cylindrical body constituting the defect hole closing material 100, is being held securely, the second end 122, which is lengthwise other end of the cylindrical body, can be pulled toward the first end 112. Thus, when the second end 122 is pulled toward the first end 112 by means of the string 140, the cylinder diameter of other section than the substantially central section 130 (the cylinder diameter of the area of the cylindrical body corresponding to the first cylindrical section 110 and the second cylindrical section 120) is increased, and also the first cylindrical section 110 and the second cylindrical section 120 are moved close to each other with respect to the substantially central section 130 serving as a center.

In the defect hole closing material 100, a porous cylindrical layer 160, which is composed of any one of a nonwoven cloth, a sponge, a film, and a composite of them that are made of a bioabsorbable material, is placed on the inner surface of the cylindrical body. The first cylindrical section 110 and the second cylindrical section 120 are each made of bioabsorbable fiber 150 knitted into a fabric, a braided textile, or a tubular-knitted fabric having a mesh-like structure as a whole. The porous cylindrical layer 160 is composed of any one of a nonwoven cloth, a sponge, a film, and a composite of them for retention of a medical agent by means of coating application, impregnation, embedding, or otherwise. Moreover, the porous cylindrical layer 160 is not limited to a bioabsorbable material, but may be of a non-bioabsorbable material.

Thus, the first cylindrical section 110, the second cylindrical section 120, the string 140 (including the string used for constriction at the substantially central section 130), and the porous cylindrical layer 160 constitute the defect hole closing material 100 of the present embodiment. All of these constituent components are made of a bioabsorbable material, wherefore the defect hole closing material 100 exhibits bioabsorbability in its entirety. Moreover, considering that the defect hole closing material 100 changes its shape within a living body, the material, the meshed sructure, the fiber structure, and the fiber sectional profile of the defect hole closing material 100 are so determined as not to cause any damage to biological tissue within the living body. Note that, under normal circumstances, the string 140 is taken out of the living body following the completion of catheterization procedure as will hereafter be described. In this regard, the string 140 does not necessarily have to possess bioabsorbability.

The bioabsorbable fiber 150 constituting the first cylindrical section 110, the second cylindrical section 120, and the string 140 (including the string used for constriction at the substantially central section 130) is at least one selected from among, for example, polyglycolic acid, polylactide (D-form, L-form, DL-form), polycaprolactone, a glycolic acid-lactide (D-form, L-form, DL-form) copolymer, a glycolic acid-ε-caprolactone copolymer, a lactide (D-form, L-form, DL-form)-ε-caprolactone copolymer, poly (p-dioxanone), and a glycolic acid-lactide (D-form, L-form, DL-form)-ε-caprolactone copolymer. While the selected material is used after being processed into any one of a monofilament yarn form, a multifilament yarn form, a twisted yarn form, a braid form, and the like, the material is preferably used in the monofilament yarn form.

The bioabsorbable fiber 150 is designed to have a diameter in an about 0.001 mm- to 1.5 mm range, and, the type of the bioabsorbable fiber and its fiber diameter are selected to suit catheterization procedure in which the defect hole closing material finds application. Moreover, the bioabsorbable fiber 150 may have any given sectional profile, for example, a circular shape, an elliptical shape, or other shape (for example, a star shape) so long as the following condition is fulfilled: biological tissue within a living body will not be impaired. Furthermore, the surface of the bioabsorbable fiber 150 may be hydrophilically treated by means of plasma discharge, electron-beam processing, corona discharge, ultraviolet irradiation, ozonation, or otherwise. In addition, the bioabsorbable fiber 150 may be subjected to application or impregnation of an X-ray impervious material (such for example as barium sulfate, a gold chip, or a platinum chip), a process of adhesion of a medical agent (for example, a medicament suitable for ASD catheterization procedure), or a process of coating of natural polymer such as collagen, gelatin, or the like or synthetic polymer such as polyvinyl alcohol, polyethylene glycol, or the like.

In forming the first cylindrical section 110 and the second cylindrical section 120, for example, the bioabsorbable fiber 150 in monofilament yarn form is knitted into a braided textile using a braider composed of a silicone-made rubber tube having a desired outer diameter (not shown in the drawings) and a plurality of yarn feeding ports (8-port or 12-port yarn feeder, for example) arranged around the tube, or is knitted into a cylindrical body of mesh-like structure having a substantially uniform diameter using a circular knitter (not shown). After the knitting operation, as has already been described, the braided textile or cylindrical body is constricted at its substantially central section 130 by a string made of the same material as that used for the first cylindrical section 110 and the second cylindrical section 120, thereby producing a construction comprising two cylindrical bodies in the form of an hourglass shape, a numeral “8” shape, a double spindle shape, or a peanut shape. The cylinder diameter of each of the first cylindrical section 110 and the second cylindrical section 120 in a diametrically contracted state is smaller than the inner diameter of a catheter, and, on the other hand, in a diametrically expanded state, the cylinder diameter stands at a level which is desirable for ASD catheterization procedure. For example, the cylinder diameter of each of the first cylindrical section 110 and the second cylindrical section 120 in a diametrically expanded state falls in the range of 5 mm to 80 mm, or preferably in the range of about 15 mm to 25 mm. Moreover, the length of each of the first cylindrical section 110 and the second cylindrical section 120, and the density of the mesh-like structure of the defect hole closing material 100 are determined to suit ASD catheterization procedure. Note that there is no need for the first cylindrical section 110 and the second cylindrical section 120 to have the same cylinder diameter and the same length, and therefore their cylinder diameters and lengths may be changed appropriately to suit ASD catheterization procedure.

There is no particular limitation to a bioabsorbable material used to form the porous cylindrical layer 160, and, exemplary of the bioabsorbable material are absorbable synthetic polymers, including: polyglycolic acid; polylactide (D-form, L-form, DL-form); polycaprolactone; a glycolic acid-lactide (D-form, L-form, DL-form) copolymer; a glycolic acid-ε-caprolactone copolymer; a lactide (D-form, L-form, DL-form)-ε-caprolactone copolymer; poly (p-dioxanone); and a glycolic acid-lactide (D-form, L-form, DL-form)-ε-caprolactone copolymer. Each of these materials can be used alone, or two or more of them can be used in combination. Particularly, it is desirable to use at least one selected from a group consisting of polyglycolic acid, a lactide (D-form, L-form, DL-form)-ε-caprolactone copolymer, a glycolic acid-ε-caprolactone copolymer, and a glycolic acid-lactide (D-form, L-form, DL-form)-ε-caprolactone copolymer from the standpoint of their moderate decomposition behaviors, and, the selected material is given the form of any one of a nonwoven cloth, a sponge, a film, and a composite of them. The form of a nonwoven cloth is particularly desirable.

The porous cylindrical layer 160, when it is formed of a nonwoven cloth, may be hydrophilically treated. There is no particular limitation to hydrophilic treatment, and, exemplary of the hydrophilic treatment are: plasma treatment, glow discharge treatment, corona discharge treatment, ozonation treatment, surface grafting treatment, and ultraviolet irradiation treatment. Among them, plasma treatment is particularly desirable, because it is conducive to significant improvement in water absorption rate without causing any change of the appearance of the nonwoven cloth. Note that the porous cylindrical layer 160 may be of either a sponge layer or a film layer, or may be of a nonwoven cloth-sponge composite layer, a nonwoven cloth-film composite layer, a sponge-film composite layer, or a composite layer of a nonwoven cloth, a sponge layer, and a film layer.

The porous cylindrical layer 160 is retentive of a medicament suitable for ASD catheterization procedure.

As thus far described, the defect hole closing material 100 of the present embodiment has the following features.

### (First Feature)

The defect hole closing material comprises the first cylindrical section 110, the second cylindrical section 120, and the substantially central section 130 which is a constricted part (a part constricted by a string made of the same material) in the form of an hourglass shape, a numeral “8” shape, a double spindle shape, or a peanut shape.

### (Second Feature)

The defect hole closing material includes the string 140 which is passed into the cylindrical body so as to be directed to and engage with the second end 122 (to be hitched on a loop at the second end 122) and then run up to the first end 112.

### (Third Feature)

The first cylindrical section 110, the second cylindrical section 120, the string 140 (including the string used for constriction at the substantially central section 130), and the porous cylindrical layer 160 constitute the defect hole closing material, and, all of these constituent components are made of a bioabsorbable material (the string 140 does not necessarily have to possess bioabsorbability).

According to the first feature and the second feature, with the first end 112 kept held securely, the second end 122 is pulled toward the first end 112 by means of the string 140, whereupon the cylinder diameter of other section than the substantially central section 130 (the cylinder diameter of the area of the cylindrical body corresponding to the first cylindrical section 110 and the second cylindrical section 120) is increased, and also the first cylindrical section 110 and the second cylindrical section 120 are moved close to each other with respect to the substantially central section 130 serving as a center.

The defect hole closing material 100 is particularly suitable for ASD catheterization procedure in that it provides the following effects.

### (First Effect)

When the second end 122 is not pulled toward the first end 112, and more specifically, when the first end 112 and the second end 122 are pulled in mutually separating directions outside of a living body, then the cylinder diameter of the defect hole closing material 100 becomes smaller than the inner diameter of a catheter, wherefore the defect hole closing material 100 can be set in the catheter.

### (Second Effect)

The defect hole closing material set in the catheter is delivered to the position of a hole developed in the atrial septum, and is then released in the atrium. Then, within the living body, while the first end 112 is being held securely, the string 140 is pulled from outside the living body so that the second end 122 can be moved toward the first end 112, whereupon the cylinder diameter of other section than the substantially central section 130 (the cylinder diameter of the area of the cylindrical body corresponding to the first cylindrical section 110 and the second cylindrical section 120) is increased, and consequently the first cylindrical section 110 located on the right-atrium side and the second cylindrical section 120 located on the left-atrium side move close to each other with respect to the substantially central section 130 serving as a center. In this way, the hole developed in the atrial septum can be closed. Moreover, upon the release of a pull from the string 140, the first cylindrical section 110 and the second cylindrical section 120 are each freed from the diametric expansion and mutual approach, and are allowed to recover their original configurations, and further, by pulling the first end 112 by means of another string, the string 140, forceps, or otherwise, the cylinder diameter of the defect hole closing material 100 becomes smaller than the inner diameter of the catheter, wherefore the defect hole closing material 100 can be set in the catheter once again, and can thus be taken out of the living body for retry of catheterization procedure.

### (Third Effect)

All of the components constituting the defect hole closing material 100 are made of a bioabsorbable material and are therefore eventually absorbed in the living body, wherefore there is little possibility of occurrence of a failure in the body in some distant future.

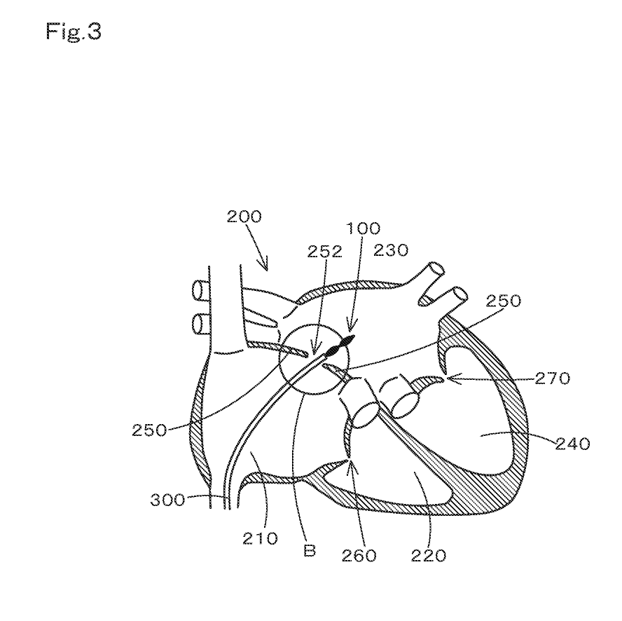

In order to simplify an understanding of such an effect, a case where the defect hole closing material 100 is used in ASD catheterization procedure will be described with reference to FIGS. 3 to 8.

### [Usage Mode]

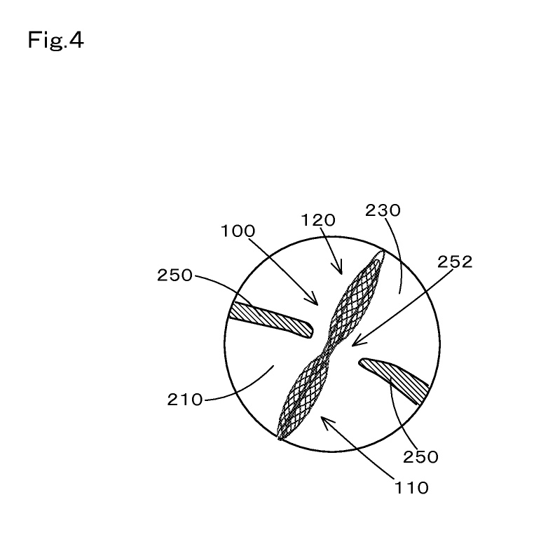

FIG. 3 shows a conceptual diagram illustrating the case of using the defect hole closing material 100 in ASD catheterization procedure, and FIGS. 4 to 8 each show an enlarged view of Part B depicted in FIG. 3, illustrating procedural steps of the ASD catheterization procedure. Note that the following description deals only with particulars specific to the mode of using the defect hole closing material 100 of the present embodiment, and a detailed explanation of other general matters will not be given, because the matters are the same as general information about heretofore known ASD catheterization procedure.

As shown in FIG. 3, human heart 200 is composed of two atria and two ventricles, namely right atrium 210 connected to superior vena cava and inferior vena cava, for receiving venous blood from all over the body; right ventricle 220 connected to the right atrium 210 via lung artery and tricuspid valve 260, for feeding venous blood to lung; left atrium 230 connected to pulmonary vein, for receiving arterial blood from lung; and left ventricle 240 connected to the left atrium 230 via aorta and mitral valve, for feeding arterial blood to the whole body. Atrial Septal Defect, or ASD is a disease characterized in that a defect hole 252 is developed in atrial septum 250 acting as a partition between the right atrium 210 and the left atrium 230. For a better understanding, in FIG. 3, there is shown a condition where the defect hole closing material 100 is left exposed from a catheter 300.

At first, in a place outside of the living body, the first end 112 and the second end 122 of the defect hole closing material 100 whose size is appropriate for the defect hole 252 are pulled in mutually separating directions, so that the cylinder diameter of the defect hole closing material 100 becomes smaller than the inner diameter of the catheter 300. Thus, the defect hole closing material 100 is set in the catheter 300. The catheter 300 is then inserted from femoral vein to allow the defect hole closing material 100 to travel from the right atrium 210 toward the left atrium 230 through the defect hole 252.

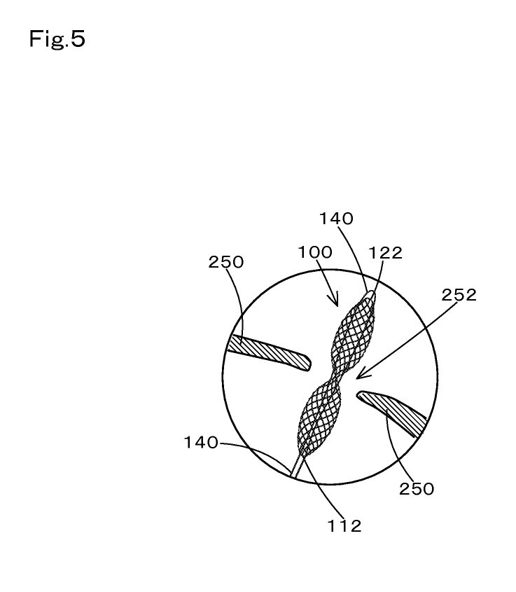

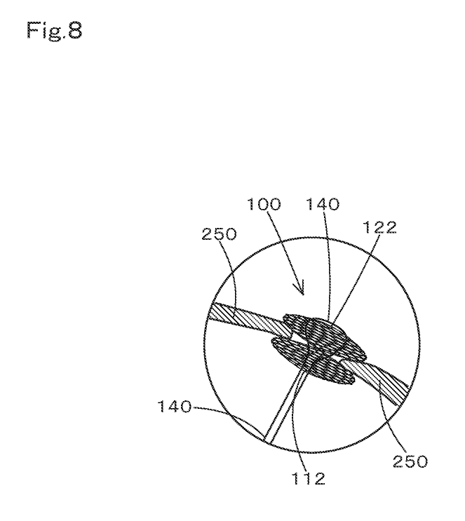

As shown in FIG. 4, the defect hole closing material 100 is brought to a stop in a position where the substantially central section 130 of the defect hole closing material 100 lies in the vicinity of the defect hole 252. Within the living body, while the first end 112 is being held securely, the string 140 is pulled from outside the living body so that the second end 122 can be moved toward the first end 112, whereupon, as shown in FIGS. 5 to 7, the cylinder diameter of other section than the substantially central section 130 (the cylinder diameter of the area of the cylindrical body corresponding to the first cylindrical section 110 and the second cylindrical section 120) is increased, and consequently the first cylindrical section 110 located on the right-atrium side and the second cylindrical section 120 located on the left-atrium side are gradually moved close to each other with respect to the substantially central section 130 serving as a center (defect hole 252), and also the first cylindrical section 110 and the second cylindrical section 120 undergo further diametric expansion. In the end, as shown in FIG. 8, the atrial septum 250 is held, at each side, by the first cylindrical section 110 and the second cylindrical section 120, thereby achieving closure of the defect hole 252 developed in the atrial septum 250 by means of the defect hole closing material 100.

In FIGS. 4 to 8, the first cylindrical section 110 and the second cylindrical section 120 are caused to undergo simultaneous diametric expansion and approaching action. Alternatively, the first cylindrical section 110 alone may be diametrically expanded first and then moved close to the second cylindrical section 120, and whereafter the second cylindrical section 120 may be diametrically expanded and then moved close to the first cylindrical section 110, or contrariwise the second cylindrical section 120 alone may be diametrically expanded first and then moved close to the first cylindrical section 110, and whereafter the first cylindrical section 110 may be diametrically expanded and then moved close to the second cylindrical section 120. In this case, the defect hole closing material can be changed in form variously with ease by, for example, shifting the position of engagement of the string 140 from the second end 122 to another area, allowing the string 140 to engage with another area in addition to the second end 122, or adding two or more strings 140.

After that, the catheter 300 and the string 140 are taken out of the living body, whereupon the catheterization procedure is completed. As a result, the defect hole closing material 100 entirely made of a bioabsorbable material is left to indwell in the living body (to be exact, in the vicinity of the defect hole 252). Since all of the components constituting the defect hole closing material 100 indwelling in the living body are made of a bioabsorbable material and are therefore eventually absorbed in the living body, it follows that there is little possibility of occurrence of a failure in the body in some distant future.

It is necessary to fix the defect hole closing material 100 in a configuration such as shown in FIG. 8 prior to indwelling of the defect hole closing material 100 in the living body. As a conceivable method for fixing the configuration of the defect hole closing material 100, for example, heat fusibility is imparted to the bioabsorbable fiber 150, so that the bioabsorbable fiber 150 can be thermally set within the living body.

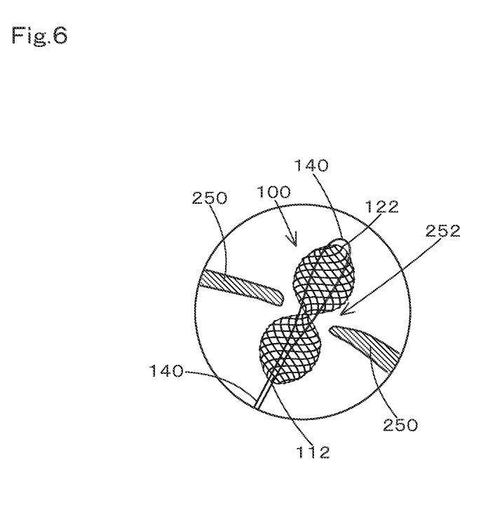

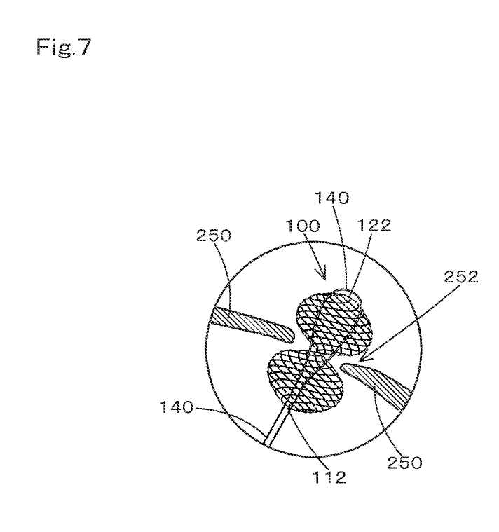

Moreover, the defect hole closing material 100 affords remarkably distinct advantages over the assembly disclosed in Patent literature 1. As has already been described, insofar as the defect hole closing material 100 is yet to be fixed in the configuration as shown in FIG. 8 within the living body, the defect hole closing material 100 can be changed from the state as shown in FIG. 8 to a state as shown in FIG. 7, from there to a state as shown in FIG. 6, and from there to a state as shown in FIG. 5, and can finally be returned to a state as shown in FIG. 4 by loosening the pulled string 140 within the living body. In addition, by pulling the first end 112 by means of another string, the string 140, forceps, or otherwise, it is possible to render the cylinder diameter of the defect hole closing material 100 smaller than the inner diameter of the catheter 300, and thereby store the defect hole closing material 100 in the catheter 300, wherefore the catheter 300 can be taken out of the living body readily, thereby facilitating retry of the catheterization procedure. For example, it is expected that catheterization procedure can be retried after making a change to the size of the defect hole closing material 100. It is apparent that the aforestated advantages cannot be attained by the assembly disclosed in Patent literature 1 that cannot be taken out of the living body without performing difficult operation using an intricate retrievable device when it is fixed in a configuration such as shown in FIG. 8 once. If the assembly disclosed in Patent literature 1 is used under such circumstances, it will be necessary to perform thoracotomy which is a highly invasive surgery.

Thus, according to the defect hole closing material 100 of the present embodiment, all of the constituent components are made of a bioabsorbable material and are therefore eventually absorbed in the living body, wherefore there is little possibility of occurrence of a failure in the body in some distant future. Moreover, the cylinder diameter of the defect hole closing material 100 is readily changeable, wherefore the defect hole closing material 100 can be set in a catheter with ease by decreasing the cylinder diameter of the defect hole closing material 100. Furthermore, a defect hole developed in the atrial septum can be closed by following a step of changing the configuration of the defect hole closing material 100 in a manner such that the cylinder diameter of the defect hole closing material 100 is increased, and the two cylindrical bodies move close to each other at the position of the defect hole, a step of fixing this configuration, and a step of indwelling the defect hole closing material in the living body. In addition, insofar as the configuration is yet to be fixed, the cylinder diameter of the defect hole closing material 100 can be readily changed within the living body, thereby permitting re-setting of the defect hole closing material 100 in a catheter, and thus facilitating retry of catheterization. This makes minimally invasive medical procedure possible.

### [Another Usage Mode]

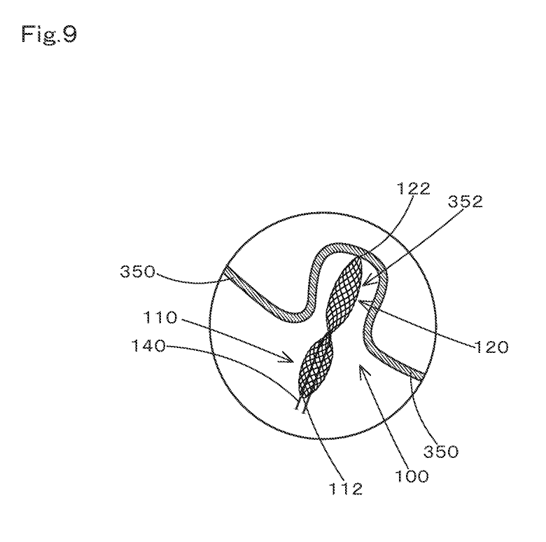

FIGS. 9 to 11 show procedural steps for a case where the defect hole closing material 100 is used in aneurysm catheterization procedure. Likewise, the following description deals only with particulars specific to another mode of using the defect hole closing material 100 of the present embodiment, and a detailed explanation of other general matters will not be given, because the matters are the same as general information about heretofore known aneurysm catheterization procedure. Moreover, an overlap with the description of the foregoing usage mode (ASD catheterization procedure) will be omitted.

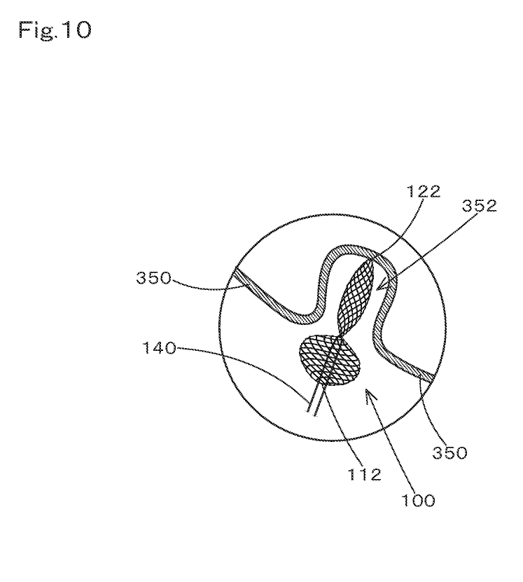

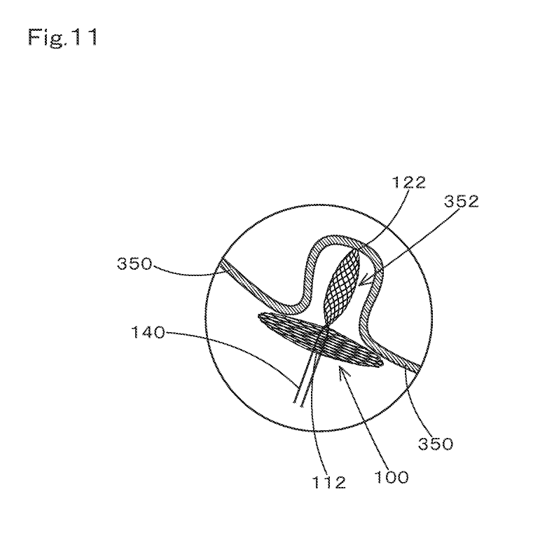

The defect hole closing material 100 includes a string 140 which is passed into the cylindrical body so as to be directed to and engage with a substantially central section 130-side end of the first cylindrical section 110 (to be hitched on a loop at the end opposite from the first end 112) and then run up to the first end 112. As shown in FIG. 9, the defect hole closing material 100 is brought to a stop in a position where the substantially central section 130 of the defect hole closing material 100 lies in the vicinity of arterial blood vessel wall 350. At this time, the second cylindrical section 120 is located in an aneurysm 352, whereas the first cylindrical section 110 is located in the artery. Then, the string 140 is operated (pulled) from outside the living body in a manner to increase the cylinder diameter of the first cylindrical section 110 while maintaining the configuration of the second cylindrical section 120 (at least restraining the second cylindrical section 120 against diametric expansion) within the living body, whereupon the cylinder diameter of the first cylindrical section 110 is increased as shown in FIG. 10. By continuing this operation, eventually, as shown in FIG. 11, the mouth area of the aneurysm 352 can be blocked by the first cylindrical section 110. This makes it possible to prevent arterial blood from flowing into the aneurysm 352.

On the other hand, it is also possible to increase the cylinder diameter of the second cylindrical section 120 while maintaining the configuration of the first cylindrical section 110 (at least restraining the first cylindrical section 110 against diametric expansion) by properly operating, from outside the living body, the string 140 which has been passed into the cylindrical body so as to engage with the second end 122 (to be hitched on a loop at the second end 122) and then run up to the first end 112 as shown in FIG. 1 which has already been cited, with the substantially central section 130 kept held securely. Depending on the position of an area to be treated within the living body, such a usage mode is considered practical.

### Modification Example

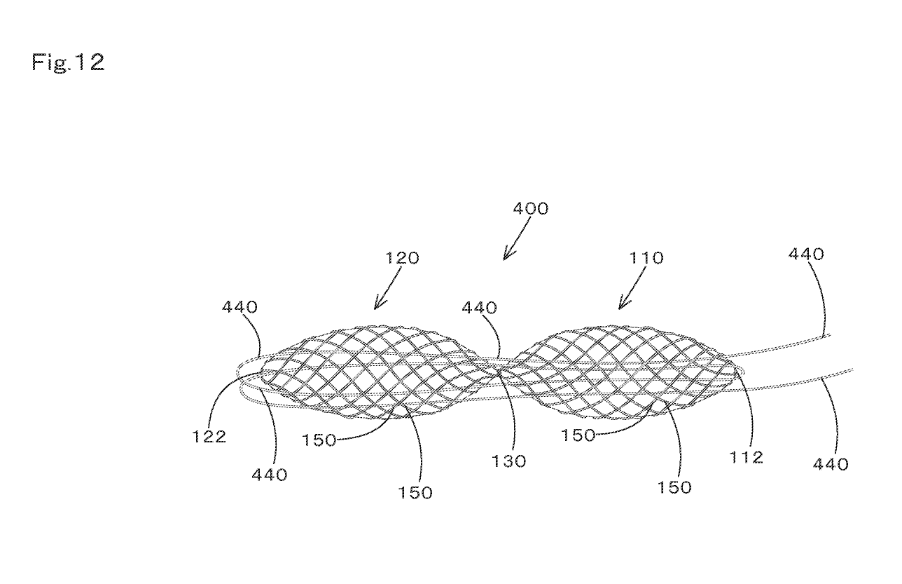

FIG. 12 shows a general perspective view of a defect hole closing material (closure plug) 400 implemented as an example of modified form of the medical material pursuant to the present invention. The defect hole closing material 400 includes a string 440 which is engaged in a manner different from the manner of engaging the string 140 of the foregoing defect hole closing material 100. Apart from the difference in string engagement, the defect hole closing material 400 is otherwise similar in constitution and workings to the defect hole closing material 100.

As shown in FIG. 12, the defect hole closing material 400 includes the string 440 which is passed into the cylindrical body from the first end 112 and is directed to the second end 122, engages with the second end 122 (is hitched on a loop at the second end 122), runs through the interior of the cylindrical body toward the first end 112, engages with the first end 112 (is hitched on a loop at the first end 112), runs through the interior of the cylindrical body once again toward the second end 122, engages with the second end 122 once again (is hitched on the loop at the second end 122 once again), and runs through the interior of the cylindrical body once again toward the first end 112. When the second end 122 is pulled toward the first end 112 by means of the string 440 (just like the way of pulling the first end 112 toward the second end 122), as is the case with the defect hole closing material 100, in the defect hole closing material 400, the cylinder diameter of other section than the substantially central section 130 (the cylinder diameter of the area of the cylindrical body corresponding to the first cylindrical section 110 and the second cylindrical section 120) is increased, and also the first cylindrical section 110 and the second cylindrical section 120 are moved close to each other with respect to the substantially central section 130 serving as a center.

In contrast to the defect hole closing material 100, in the defect hole closing material 400, the configuration of the defect hole closing material 400 can be maintained simply by pulling the string 440 (without the necessity of securing the string 440). Thus, in the defect hole closing material 400, its configuration can be maintained easily without the necessity of securing the string 440.

In the defect hole closing material 400, instead of engaging the string 440 in double-loop form with the second end 122 (hitching it on a loop at the second end 122), by engaging the string 440 with a substantially central section 130-side end of the second cylindrical section 120 (hitching it on a loop at the end opposite from the second end 122), as has already been described with reference to FIGS. 9 to 11, it is possible to increase the cylinder diameter of the first cylindrical section 110 while maintaining the configuration of the second cylindrical section 120 (at least restraining the second cylindrical section 120 against diametric expansion), as well as to maintain the configuration of the defect hole closing material 400 easily without the necessity of securing the string 440.

Contrariwise, in the defect hole closing material 400, instead of engaging the string 440 in single-loop form with the first end 112 (hitching it on a loop at the first end 112), by engaging the string 440 with a substantially central section 130-side end of the first cylindrical section 110 (hitching it on a loop at the end opposite from the first end 112), it is possible to increase the cylinder diameter of the second cylindrical section 120 while maintaining the configuration of the first cylindrical section 110 (at least restraining the first cylindrical section 110 against diametric expansion) as has already been described with reference to FIGS. 9 to 11 (to be exact, the target cylindrical section to be diametrically expanded is the reverse of that indicated in FIGS. 9 to 11), as well as to maintain the configuration of the defect hole closing material 400 without the necessity of securing the string 440.

In many cases, the string 440 of the defect hole closing material 400 is not taken out of the living body even after the completion of catheterization procedure. In such a case, the string 440 must possess bioabsorbability by necessity.

It should be understood that the embodiments as set forth hereinabove are considered in all respects as illustrative only and not restrictive. The scope of the present invention is indicated by the appended claims rather than the foregoing description, and all changes that come within the meaning of and the range of equivalency of the claims are intended to be embraced therein.

### INDUSTRIAL APPLICABILITY

The present invention is suitable for use as a medical material which is set in a catheter for a treatment of a physical defect (hole, lump) developed in biological tissue, and the medical material embodying the present invention is particularly desirable in that it is releasable and is able to indwell in a target body area to be treated, allows minimally invasive medical procedure, and poses little possibility of occurrence of a failure in the body in some distant future even if it remains in the body.

### EXPLANATION OF REFERENCE SYMBOLS

100, 400 defect hole closing material (closure plug)
110 first cylindrical section
112 first end
120 second cylindrical section
122 second end
130 substantially central section
140, 440 string
150 bioabsorbable fiber
160 porous cylindrical layer
200 heart
250 atrial septum
252 defect hole
300 catheter
350 (arterial) blood vessel wall
352 aneurysm

1. A medical material which comprises a cylindrical body of mesh-like structure made of a filamentary material, and has a configuration such that a cylinder diameter of a substantially central section of said cylindrical body is made smaller than a cylinder diameter of other section of said cylindrical body by constricting said substantially central section.

2. The medical material according to claim 1,
wherein said configuration is defined by an hourglass shape, a numeral “8” shape, or a double spindle shape.

3. The medical material according to claim 1,
wherein said filamentary material is a bioabsorbable material.

4. The medical material according to claim 1,
wherein a porous cylindrical layer, which is composed of any one of a nonwoven cloth, a sponge, a film, and a composite of them that are made of a bioabsorbable material, is placed on an inner surface of said cylindrical body.

5. The medical material according to claim 1,
wherein the cylinder diameter of said other section can be increased by, while securely holding a first end which is one end of said medical material in a direction of length of the cylindrical body, pulling a second end, which is the other end of said medical material, toward the first end.

6. The medical material according to claim 5, further comprising:
a string which is passed into said cylindrical body so as to be directed to and engage with said second end, and then run up to said first end.

7. The medical material according to claim 1,
wherein, with respect to said substantially central section serving as a center, there are provided a first cylindrical section located toward the first end and a second cylindrical section located toward the second end,
and wherein said first cylindrical section and said second cylindrical section are caused to move close to each other with respect to said substantially central section serving as a center by pulling the second end, or equivalently the other end of said cylindrical body toward the first end of said cylindrical body while holding the first end securely.

8. The medical material according to claim 7, further comprising:
a string which is passed into said cylindrical body so as to be directed to and engage with said second end, and then run up to said first end.

9. The medical material according to claim 1,
wherein, with respect to said substantially central section serving as a center, there are provided a first cylindrical section located toward the first end and a second cylindrical section located toward the second end,
and wherein a cylinder diameter of said first cylindrical section can be increased by, while securely holding the first end of said cylindrical body, pulling a substantially central section-side end of said first cylindrical section toward the first end.

10. The medical material according to claim 9, further comprising:
a string which is passed into said cylindrical body so as to be directed to and engage with the substantially central section-side end of said first cylindrical section, and then run up to said first end.

11. The medical material according to claim 1,
wherein, with respect to said substantially central section serving as a center, there are provided a first cylindrical section located toward the first end and a second cylindrical section located toward the second end,
and wherein there is provided a string which is passed into said cylindrical body from said first end, is directed to said second end, engages with said second end, runs through an interior of said cylindrical body toward said first end, engages with said first end, runs through the interior of said cylindrical body once again toward said second end, engages with said second end once again, and runs through the interior of the cylindrical body once again toward said first end.

12. The medical material according to claim 11,
wherein said configuration is defined by an hourglass shape, a numeral “8” shape, or a double spindle shape.

13. The medical material according to claim 11,
wherein said filamentary material is a bioabsorbable material.

14. The medical material according to claim 11,
wherein a porous cylindrical layer, which is composed of any one of a nonwoven cloth, a sponge, a film, and a composite of them that are made of a bioabsorbable material, is placed on the inner surface of said cylindrical body.

15. The medical material according to claim 1,
wherein, with respect to said substantially central section serving as a center, there are provided a first cylindrical section located toward the first end and a second cylindrical section located toward the second end,
and wherein there is provided a string which is passed into said cylindrical body from said first end, is directed to said second end, engages with said second end, runs through the interior of said cylindrical body toward said substantially central section, engages with said substantially central section, runs through the interior of said cylindrical body once again toward said second end, engages with said second end once again, and runs through the interior of the cylindrical body once again toward said first end.

16. The medical material according to claim 15,
wherein said configuration is defined by an hourglass shape, a numeral “8” shape, or a double spindle shape.

17. The medical material according to claim 15,
wherein said filamentary material is a bioabsorbable material.

18. The medical material according to claim 15,
wherein a porous cylindrical layer, which is composed of any one of a nonwoven cloth, a sponge, a film, and a composite of them that are made of a bioabsorbable material, is placed on the inner surface of said cylindrical body.
